(19) United States
(12) Patent Application Publication
(10) Pub. No.: US 2006/0127312 A1
(43) Pub. Date: Jun. 15, 2006

(54) Notch signaling in long-term memory formation

(75) Inventors: Yi Zhong, Huntington, NY (US); Xuecai Ge, Boston, MA (US); Zuoping Xie, Beijing (CN)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Cold Spring Harbor Laboratory, Cold Spring Harbor, NY (US)

(21) Appl. No.: 11/173,899

(22) Filed: Jul. 1, 2005

Related U.S. Application Data
(60) Provisional application No. 60/585,331, filed on Jul. 2, 2004.

(51) Int. Cl. A61K 49/00; A61K 48/00; A61K 38/17

(52) U.S. Cl. 424/9.2; 514/12; 514/44

(57) ABSTRACT

The present invention is directed to a method of modulating (e.g., enhanced, increased) long term memory formation in an animal comprising modulating Notch function (e.g., Notch protein expression) in said animal. In addition, the present invention is directed to a method of treating an animal with a defect in long term memory associated with a defect in Notch comprising increasing Notch function in the animal relative to Notch function in the animal prior to the treatment. The present invention is also directed to a method of preventing a disease condition in an animal where long term memory is diminished wherein said disease condition is caused by loss of Notch function, by administering a therapeutic amount of Notch protein to an animal in need thereof. Methods for identifying a pharmaceutical agent for its ability to modulate Notch function are also encompassed by the invention.

### RELATED APPLICATIONS

This application claims the benefit of U.S. Provisional Application No. 60/585,331, filed Jul. 2, 2004. The entire teachings of the above application are incorporated herein by reference.

### BACKGROUND OF THE INVENTION

Notch (N) is a cell surface receptor that mediates an evolutionarily ancient signaling pathway to control an extraordinarily broad spectrum of cell fates and developmental processes. Activation of N receptors has been linked to the specification of many cell types in both vertebrates and invertebrates (Artavanis-Tsakonas, S., et al., Science, 284:770-776 (1999)). Binding of ligands such as Delta (Parks, A. L., et al., Mech. Dev., 50:201-216 (1995)) causes cleavage of the intracellular domain of the N protein (Kidd, S., et al., Genes Dev., 12:3728-3740 (1998); Schroeter, E. H., et al., Nature, 393:382-386 (1998); Struhl, G., et al., Cell, 93:649-660 (1998)). The cleaved cytoplasmic domain of N (Nintra) enters the nucleus, in which it regulates expression of target genes (Artavanis-Tsakonas, S., et al., Science, 284:770-776 (1999)). Intercellular communication mediated by N signaling consists of two different modes: lateral inhibitory signaling and inductive signaling. A prototypic example of lateral inhibitory signaling is that loss of N function causes a cluster of equivalent proneural cells to all assume the default neuronal fate rather than an epidermal fate (Artavanis-Tsakonas, S., et al., Science, 268:225-232 (1995)). Inductive N signaling, on the other hand, mediates interactions between cells that are nonequivalent before the signal initiates, such as induction of cone cells by photoreceptor cells and specification of midline cells in the embryo (Flore, G., et al., Cell, 103:75-85 (2000); Lecourtois, M., et al., Genes Dev., 9:2598-2608 (1995); Morel, V., et al., Curr. Biol., 11: 789-792 (2001)).

In contrast to the extensive understanding of the vital role of N in development, the significance of N signaling in adult brains has yet to be revealed, although there is continuous presence of N protein and its ligands in the adult vertebrate nervous system (Stump, G., et al., Mech Dev., 114:153-159 (2002)).

### SUMMARY OF THE INVENTION

To gain insights into the functions of N signaling in the adult brain, the involvement of N in Drosophila olfactory learning and memory was examined. Long-term memory (LTM) was disrupted by blocking N signaling in conditional mutants or by acutely induced expression of a dominant-negative N transgene. In contrast, neither learning nor early memory were affected. Furthermore, induced overexpression of a wild-type (normal) N transgene specifically enhanced LTM formation. These experiments demonstrate that N signaling contributes to LTM formation in the Drosophila adult brain.

Accordingly, the present invention is directed to a method of modulating (e.g., enhanced, increased) long term memory formation in an animal comprising modulating Notch function (e.g., Notch protein expression) in said animal. Modulating Notch function can comprise administering to the animal a pharmaceutical agent which modulates Notch function in the animal, in an amount effective to modulate Notch function in the animal (e.g., a rodent or a human). In one embodiment, modulation of Notch protein expression comprises administering to the animal a pharmaceutical agent which modulates Notch protein expression in the animal, in an amount effective to modulate Notch protein expression in the animal.

The present invention is also directed to a method of enhancing long term memory formation in an animal comprising treating the animal to increase expression of Notch gene relative to expression of the Notch gene in the animal prior to said treatment.

In addition, the present invention is directed to a method of treating an animal with a defect in long term memory associated with a defect in Notch comprising increasing Notch function in the animal relative to Notch function in the animal prior to the treatment. In one embodiment, increasing Notch function comprises administering to the animal a pharmaceutical agent which increases Notch function in the animal, in an amount effective to increase Notch function relative to Notch function in the animal prior to the administering of the pharmaceutical agent. Notch protein expression can be increased by administering to the animal a pharmaceutical agent which increases Notch protein expression in the animal, in an amount effective to increase Notch protein expression relative to Notch protein expression in the animal prior to treatment. In a particular embodiment, an effective amount of a composition comprising Notch, a Notch analog, biologically active Notch fragment or a Notch fusion protein is administered. In yet another embodiment, an effective amount of a composition comprising a nucleic acid sequence which encodes Notch, a Notch analog, biologically active Notch fragment or a Notch fusion protein is administered.

The present invention is also directed to a method of treating an animal with a defect in long term memory associated with a defect in Notch gene function comprising treating the animal to increase expression of Notch gene relative to expression of the Notch gene in the animal prior to said treatment.

Also encompassed by the present invention is a method of preventing a disease condition in an animal where long term memory is diminished wherein said disease condition is caused by loss of Notch function, by administering a therapeutic amount of Notch protein to an animal in need thereof. In one embodiment, an inducible form of the Notch gene or an inducible homolog thereof is administered to the animal in need thereof. In a particular embodiment, Notch protein expression is increased in the animal relative to Notch protein expression in the animal prior to treatment.

The present invention is also directed to a method for identifying a pharmaceutical agent for its ability to modulate Notch function comprising introducing a pharmaceutical agent of interest into host cells expressing Notch; and determining Notch function in the host cells. A difference in Notch function compared to Notch function of the host cells to which the pharmaceutical agent has not been introduced identifies the pharmaceutical agent as a pharmaceutical agent that modulates Notch function. In one embodiment, the host cells express Notch as a Notch::indicator fusion protein. In another embodiment, Notch function is determined by detecting and determining the level of Notch::indicator fusion protein mRNA produced. In yet another embodiment, Notch function is determined by detecting and determining the level of Notch::indicator fusion protein produced. Notch function can also be determined by detecting the presence of the cleaved cytoplasmic domain of the Notch in the host cells' nucleus. Alternatively, Notch function can be determined by measuring downstream products regulated by the Notch gene product.

The method of screening the pharmaceutical agent for its ability to modulate long term memory formation in an animal can further comprise administering the pharmaceutical agent identified to an animal; and training the animal under conditions appropriate to produce long term memory in the animal. Long term memory in the animal trained is assessed; and compared to long term memory produced in a control animal to whom the pharmaceutical agent has not been administered and who has been trained, wherein the pharmaceutical agent is able to modulate long term memory formation in an animal if long term memory formation in the animal is altered when compared to the control animal.

The invention is also directed to a method of screening a pharmaceutical agent for its ability to modulate long term memory formation in an animal comprising administering the pharmaceutical agent to the animal; and determining the functional Notch gene activity in the animal obtained relative to the functional Notch gene activity in a control animal to whom said pharmaceutical agent has not been administered. The animal having a functional Notch gene activity which differs from the functional Notch gene activity in the control animal to whom said pharmaceutical agent has not been administered is selected; and trained under conditions appropriate to produce long term memory in said animal. Long term memory in the animal trained is assessed; and compared to long term memory produced in the control animal to whom the pharmaceutical agent has not been administered, wherein the pharmaceutical agent is able to modulate long term memory formation in an animal if long term memory in the animal is altered when compared to the control animal to whom the pharmaceutical agent has not been administered.

The present invention is also directed to a method of screening for or identifying a pharmaceutical agent capable of modulating Notch function comprising introducing a pharmaceutical agent of interest into host cells expressing a Notch::indicator fusion protein; and determining Notch function. A difference in the Notch function compared to the Notch function of host cells to which said pharmaceutical agent has not been administered identifies the pharmaceutical agent as one capable of modulating Notch function.

### BRIEF DESCRIPTION OF THE DRAWINGS

FIGS. 1A-1D are graphs showing blockade of LTM formation by disruption of N signaling. FIG. 1A shows learning is not affected in temperature-sensitive N mutants. Learning scores were obtained at permissive (18° C.) and restrictive (30° C.) temperatures (see Materials and Methods) for the temperature-sensitive N mutant, N^{ts2} and for control flies. In this and all following figures, the isogenic line, w¹¹¹⁸ (isoCj1) (Yin, J., et al., Cell, 79:49-58 (1994)) was used as a control (n−7, 2, 8, and 7 for data points from left to right). FIG. 1B shows reduced 24-h memory at the restrictive temperature for Nts². Twenty-four-hour memory was determined after spaced training (10 training sessions with a 15-min rest interval between each). Spaced training induces formation of persistent memory that consists of two distinct components: protein synthesis-dependent LTM and ARM (n=4 for all data points). FIG. 1C shows learning is not affected by acutely induced expression of a dominant-negative N transgene (N^{Δcdc10rpts}) Expression of N^{Δcdc10rpts} was induced by 37° C. heat shock for 1 h followed by 3 h of rest before training (n=6 for all data points). FIG. 1D shows blockade of LTM but not ARM by acutely induced expression of dominant-negative N^{Δcdc10rpts}. The LTM component of persistent memory induced by spaced training is blocked, but ARM induced by massed (or spaced) training is not affected (n=8, 10, 8, 8, 15, 15, 17, and 17 for data points from left to right).

FIGS. 2A-2B show that learning is not affected by overexpression of wild-type N⁺ (hs-N⁺) using flies subjected to 30 min of heat shock at 37° C. followed by 3 h of rest. FIG. 2A is a graph showing learning scores were similar for controls and hs-N⁺ flies regardless of whether they were subjected to heat-shock treatment (n=2, 4, 2, and 4 for data points from left to right). FIG. 2B is a gel showing heat shock induced expression of hs-N⁺ cDNA in adult heads. Semiquantitative RT-PCR using N primer pairs N1-N2, N3-N4, and N5-N6 shows induction of the hs-N′ transgene by 30 min of heat shock at 37° C. followed by 3 h of rest (lane C) when compared with PCR from control flies that were kept at 18° C. (lane A) or 25° C. (lane B). The rp49F-R control primers show no temperature-induced increase in expression of rp49. Details of PCR primer pairs and expected products are given in Materials and Methods. Three separate mRNA isolations showed the same pattern of increased expression of the hs-N⁺ transgene after 37° C. heat shock.

FIGS. 3A-3B are graphs showing LTM is enhanced by overexpression of wild-type N⁺ (hs-N⁺). FIG. 3A is a graph showing enhanced LTM after acutely induced overexpression of wild-type N^{+.} Flies were subjected to the same heat-shock treatment as described above. Twenty-four-hour memory was compared between hs-N⁺ flies with and without heat shock for 1, 2, and 10 spaced training cycles, respectively (n=6, 6, 5, 7, 7, 9, 5, 5, 6, 6, 7, and 8 for data points from left to right). FIG. 1B is a graph showing blockade of enhanced LTM by an inhibitor of protein synthesis (cycloheximide). Flies were subjected to the same heat-shock treatment as described for FIG. 3A Twenty-four-hour memory was compared between hs-N⁺ flies with and without heat shock for one and two spaced training cycles, respectively (n=7, 9, 5, and 5 for data points from left to right; see Materials and Methods for drug-feeding conditions.)

### DETAILED DESCRIPTION OF THE INVENTION

It has been reported that N signaling regulates neurite outgrowth in mammals (Sestan, N., et al., Science, 286:741-746 (1999)), and chronic reduction of N activity in adult fruit flies also leads to progressive neurological syndromes (Presente, A. et al., NeuroReport, 12:3321-3325 (2001)). Processing of N requires γ-secretase activity, whereas presenilin (PS) is a critical component of the γ-secretase complex (Sisodia, S. S., e al., Nat. Rev. Neurosci., 3:281-290 (2002)). Mutations in the PS genes are associated with the early onset of Alzheimer's disease (Price, D., et al., Annu. Rev., Genet., 32:461-493 (1998)). Conditional knockout of the PS1 gene in mice is also associated with reduced clearance of hippocampal memory traces (Feng, R., et al., Neuron, 32:911-926 (2001)). Although involvement of N in these PS mutant phenotypes remains to be determined, it has been shown that N plays a role in PS-mediated formation of neural projections in postmitotic neurons necessary for learned thermotaxis in Caenorhabditis elegans (Wittenberg, N., et al., Nature, 406:306-309 (2000)). As described herein, the role of N signaling in learning and memory has been examined using Drosophila.

A Pavlovian procedure that pairs odors with foot-shock (Tullu, T., et al., J. Comp. Physiol., 157:263-277 (1985)) was used in this study to assess the effects of N signaling on adult behavioral plasticity. Genetic analyses have demonstrated that memory formation after such Pavlovian training occurs in functionally distinct temporal phases (Tully, T., et al., Cell, 79:35-47 (1994)). Two of these memory phases, short-term memory and middle-term memory, are labile and short-lived, whereas another two phases, anesthesia-resistant memory (ARM) and long-term memory (LTM), are resistant to various disruptive treatments and persist for several days. LTM and ARM have been dissected genetically. Disruptions of two transcription factors, cAMP-response element-binding protein (CREB) or alcohol dehydrogenase factor-1, abolish LTM without affecting ARM (Yin, J. C., et al., Cell, 79:49-58 (1994); DeZazzo, J., et al., Neuron, 27:145-158 (2000)). Conversely, ARM is disrupted but LTM is normal in radish mutants and in transgenic flies expressing a dominant-negative form of atypical protein kinase C designated PKM (Tully, T., et al., Cell, 79:35-47 (1994); Drier, E. A., et al., Nat. Neurosci., 5:316-324 (2002); Chiang, A.-S., Curr. Biol., 14:263-272 (2004)). The experiments described herein focused on LTM formation in adult flies carrying a conditional mutation of N or expressing N transgenes.

Accordingly the invention relates to methods of modulating long term memory formation in an animal. In a particular embodiment, the animal is an adult mammal. In one embodiment, the method comprises treating the animal to modulate Notch (e.g., Notch-dependent) protein expression. In a second embodiment, the method comprises treating the animal to modulate Notch function. In a particular embodiment, the method comprises administering to the animal an effective amount of a pharmaceutical agent which modulates Notch function in the animal. In another embodiment, the method comprises treating the animal to modulate Notch protein expression. In a particular embodiment, the method comprises administering to the animal an effective amount of a pharmaceutical agent which modulates Notch protein expression in the animal.

The present invention also relates to methods of enhancing long term memory formation in an animal, preferably an adult mammal. In one embodiment, the method comprises treating the animal to modulate Notch (e.g., Notch-dependent) protein expression. In a second embodiment, the method comprises treating the animal to increase Notch function relative to the Notch function in the animal prior to treatment. In a particular embodiment, treatment to increase Notch function comprises administering to the animal an effective amount of a pharmaceutical agent which increases Notch function relative to Notch function in the animal prior to administration of the pharmaceutical agent. In a second embodiment, the method comprises treating the animal to increase Notch protein expression relative to Notch protein expression in the animal prior to treatment. In a particular embodiment, treatment to increase Notch protein expression comprises administering to the animal an effective amount of a pharmaceutical agent which increases Notch protein expression relative to Notch protein expression in the animal prior to administration of the pharmaceutical agent. In another embodiment, the method comprises administering to the animal an effective amount of a Notch molecule, Notch analog, biologically active Notch fragment or Notch fusion protein. In still another embodiment, the method comprises administering to the animal an effective amount of a nucleic acid sequence encoding a Notch molecule, Notch analog, biologically active Notch fragment or Notch fusion protein.

The present invention further provides methods for treating an animal with a defect in long term memory formation associated with a defect in Notch. The animal is preferably an adult mammal. The defect in Notch is either a diminution in the amount of Notch produced, a diminution in Notch function of Notch produced or both a diminution in amount of Notch produced and Notch function of Notch produced. In one embodiment, the method comprises treating an animal with a defect in long term memory formation associated with a defect in Notch to increase Notch function relative to the Notch function in the animal prior to treatment. In a particular embodiment, treatment to increase Notch function comprises administering to the animal an effective amount of a pharmaceutical agent which increases Notch function relative to Notch function in the animal prior to administration of the pharmaceutical agent. In a second embodiment, the method comprises treating an animal with a defect in long term memory formation associated with a defect in Notch to increase Notch protein expression relative to Notch protein expression in the animal prior to treatment. In a particular embodiment, treatment to increase Notch protein expression comprises administering to the animal an effective amount of a pharmaceutical agent which increases Notch protein expression relative to Notch protein expression in the animal prior to administration of the pharmaceutical agent. In a another embodiment, the method comprises administering to an animal with a defect in long term memory formation associated with a defect in Notch a Notch compound such as exogenous Notch, Notch analog, biologically active Notch fragment or Notch fusion protein. In another embodiment, the method comprises administering to an animal with a defect in long term memory formation with a defect in Notch a nucleic acid sequence encoding Notch, Notch analog, biologically active Notch fragment or Notch fusion protein.

Notch analogs, or derivatives, are defined herein as proteins having amino acid sequences analogous to the Notch gene products described herein. Analogous amino acid sequences are defined herein to mean amino acid sequences with sufficient identity of amino acid sequence of the Notch gene product described herein to possess the biological activity or biological function or action of the Notch gene product, but with one or more “SILENT” changes in the amino acid sequence.

Biologically active Notch fragments refer to biologically active polypeptide fragments of Notch and can include only a part of the full-length amino acid sequence of Notch, yet possess biological activity of Notch (e.g., a Notch fragment that includes the intracellular domain that diffuses into the nucleus after Notch activation and thereby regulates gene expression in the Notch pathway). Such fragments can be produced by carboxyl or amino terminal deletions, as well as one or more internal deletions.

Notch fusion proteins comprise Notch as described herein, referred to as a first moiety, linked to a second moiety not occurring in the Notch protein. The second moiety can be a single amino acid, peptide or polypeptide (e.g., a heat shock protein) or other organic moiety, such as a carbohydrate, a lipid or an inorganic molecule.

The present invention further encompasses biologically active derivatives or analogs of Notch, referred to herein as Notch peptide mimetics. These mimetics can be designed and produced by techniques known to those skilled in the art. See, e.g., U.S. Pat. Nos. 5,643,873 and 5,654,276. These mimetics are based on Notch sequences. Notch sequences are readily available in the art (see, e.g., Artavanis-Tsakonas et al., PNAS, 80(7):1977-1981 (1983); GenBank Accession No. NM_{—}057511; Kidd, S., et al., Mol. Cell Biol., 6(9):3094-3108 m(1986); GenBank Accession No. K03508; Ellison, L. W., et al., Cell, 66(4):649-661 (1991); GenBank Accession No. M73980). Peptide mimetics can possess biological activity or biological function or action similar to the biological activity or biological function or action of the corresponding peptide compound, and also possess a “biological advantage” over the corresponding peptide inhibitor with respect to one, or more, of the following properties: solubility, stability and susceptibility to hydrolysis and proteolysis.

Methods for preparing peptide mimetics include modifying the N-terminal amino group, the C-terminal carboxyl group and/or changing one or more of the amino linkages in the peptide to a non-amino linkage. Two or more such modifications can be coupled in one peptide mimetic. Examples of modifications of peptides to produce peptide mimetics are described in U.S. Pat. Nos. 5,643,873 and 5,654,276.

Increased Notch protein expression or production can be achieved by administration of an exogenous Notch or, alternatively, by increasing production of the endogenous Notch, for example by stimulating the endogenous gene to produce increased amounts of Notch as known in the art. In a preferred embodiment, suitable pharmaceutical agents, as described herein, can be administered to the animal to stimulate the endogenous gene to produce increased amounts of a functional Notch, thereby increasing Notch function in the animal.

In some animals, the amount of Notch being produced can be of sufficient quantity, but the Notch is abnormal in some way and, thus, cannot exert its biological effect at all or cannot exert its biological effect to the full extent. That is, the Notch being produced has diminished or no functional activity (e.g., no biological activity, function or action). In this instance, providing copies of normal Notch genes to the animal using techniques of gene transfer well known to those skilled in the art, can increase Notch function or concentration in the animal. In another embodiment, an exogenous Notch, Notch analog, biologically active Notch fragment or Notch fusion protein can be administered to the animal.

Nucleic acid sequences are defined herein as heteropolymers of nucleic acid molecules. The nucleic acid molecules can be double stranded or single stranded and can be a deoxyribonucleotide (DNA) molecule, such as cDNA or genomic DNA, or a ribonucleotide (RNA) molecule. As such, the nucleic acid sequence can, for example, include one or more exons, with or without, as appropriate, introns, as well as one or more suitable control sequences. In one example, the nucleic acid molecule contains a single open reading frame which encodes a desired nucleic acid product. The nucleic acid sequence can be “operably linked” to a suitable promoter.

A nucleic acid sequence encoding a desired Notch, Notch analog, biologically active Notch fragment or Notch fusion protein can be isolated from nature, modified from native sequences or manufactured de novo, as described in, for example, Ausubel et al., Current Protocols in Molecular Biology, (New York: John Wiley & Sons) (1998); and Sambrook et al., Molecular Cloning: A Laboratory Manual, 2nd edition (New York: Cold Spring Harbor University Press) (1989). Nucleic acids can be isolated and fused together by methods known in the art, such as exploiting and manufacturing compatible cloning or restriction sites.

Typically, the nucleic acid sequence will be a gene which encodes the desired Notch, Notch analog or Notch fusion protein. Such a gene is typically operably linked to suitable control sequences capable of effecting the expression of the Notch, preferably in the CNS. The term “operably linked”, as used herein, is defined to mean that the gene (or the nucleic acid sequence) is linked to control sequences in a manner which allows expression of the gene (or the nucleic acid sequence). In one embodiment, operably linked means contiguous.

Control sequences include a transcriptional promoter, an optional operator sequence to control transcription, a sequence encoding suitable messenger RNA (mRNA) ribosomal binding sites and sequences which control termination of transcription and translation. In a particular embodiment, a recombinant gene (or a nucleic acid sequence) encoding Notch, Notch analog, biologically active Notch fragment or Notch fusion protein can be placed under the regulatory control of a promoter, which can be induced or repressed, thereby offering a greater degree of control with respect to the level of the product.

As used herein, the term “promoter” refers to a sequence of DNA, usually upstream (5′) of the coding region of a structural gene, which controls the expression of the coding region by providing recognition and binding sites for RNA polymerase and other factors which may be required for initiation of transcription. Suitable promoters are well known and readily available in the art (see, e.g., Ausubel et al., Current Protocols in Molecular Biology (New York: John Wiley & Sons, Inc.) (1998); Sambrook et al., Molecular Cloning: A Laboratory Manual, 2nd edition (New York: Cold Spring Harbor University Press (1989); and U.S. Pat. No. 5,681,735).

Notch, Notch analogs, biologically active Notch fragments, Notch fusion proteins and pharmaceutical agents, as well as nucleic acid sequences encoding Notch, Notch analogs, biologically active Notch fragments or Notch fusion proteins can be administered directly to an animal in a variety of ways. In a particular embodiment, administration is via transplant of neural tissue, e.g., by injecting neural cells into the brain. Other routes of administration are generally known in the art and include intravenous injection including infusion and/or bolus injection, intracerebroventricular, intrathecal, parenteral, mucosal, implant, intraperitoneal, oral, intradermal, transdermal (e.g., in slow release polymers), intramuscular, subcutaneous, topical, epidural, etc. routes. Other suitable routes of administration can also be used, for example, to achieve absorption through epithelial or mucocutaneous linings. Notch, Notch analogs, biologically active Notch fragments and Notch fusion proteins can also be administered by gene therapy, wherein a DNA molecule encoding a particular therapeutic protein or peptide is administered to the animal, e.g., via a vector, which causes the particular protein or peptide to be expressed and secreted at therapeutic levels in vivo.

A nucleic acid sequence encoding a protein or peptide (e.g., Notch, Notch analog, biologically active Notch fragment or Notch fusion protein) can be inserted into a nucleic acid vector according to methods generally known in the art (see, e.g., Ausubel et al., eds., Current Protocols in Molecular Biology, John Wiley & Sons, Inc., New York (1998); Sambrook et al., eds., Molecular Cloning: A Laboratory Manual, 2nd edition (New York: Cold Spring Harbor University Press) (1989)).

The mode of administration is preferably at the location of the target cells. In a particular embodiment, the mode of administration is to cells of neural origin. Cells of neural origin include neural stem cells, neuroblastoma cells and neurons.

Notch, Notch analogs, biologically active Notch fragments, Notch fusion proteins and pharmaceutical agents, as well as nucleic acid sequences encoding Notch, Notch analogs, biologically active Notch fragments or Notch fusion proteins can be administered together with other components of biologically active agents, such as pharmaceutically acceptable surfactants (e.g., glycerides), excipients (e.g., lactose), stabilizers, preservatives, humectants, emollients, antioxidants, carriers, diluents and vehicles. If desired, certain sweetening, flavoring and/or coloring agents can also be added.

Notch, Notch analogs, biologically active Notch fragments, Notch fusion proteins and pharmaceutical agents, as well as nucleic acid sequences encoding Notch, Notch analogs, biologically active Notch fragments or Notch fusion proteins can be administered prophylactically or therapeutically to an animal prior to, simultaneously with or sequentially with other therapeutic regimens or agents (e.g., multiple drug regimens), including with other therapeutic regimens used for the treatment of long term memory defects, or the enhancement of long term memory formation. Notch, Notch analogs, biologically active Notch fragments, Notch fusion proteins and pharmaceutical agents, as well as nucleic acid sequences encoding Notch, Notch analogs, biologically active Notch fragments or Notch fusion proteins, that are administered simultaneously with other therapeutic agents can be administered in the same or different compositions. Two or more different Notch, Notch analogs, biologically active Notch fragments, Notch fusion proteins, nucleic acid sequences, pharmaceutical agents or combinations thereof can also be administered.

Notch, Notch analogs, biologically active Notch fragments, Notch fusion proteins, and pharmaceutical agents, as well as nucleic acid sequences encoding Notch, Notch analogs, biologically active Notch fragments or Notch fusion proteins, can be formulated as a solution, suspension, emulsion or lyophilized powder in association with a pharmaceutically or physiologically acceptable parenteral vehicle, carrier or excipient. Examples of such vehicles, carriers and excipients are water, saline, Ringer's solution, isotonic sodium chloride solution, dextrose solution, and 5% human serum albumin. Liposomes and nonaqueous vehicles such as fixed oils can also be used. The vehicle or lyophilized powder can contain additives that maintain isotonicity (e.g., sodium chloride, mannitol) and chemical stability (e.g., buffers and preservatives). The formulation can be sterilized by commonly used techniques. Suitable pharmaceutical carriers are described in Remington's Pharmaceutical Sciences.

An used herein “a therapeutic amount”, “an effective amount” and “a therapeutically effective amount” of a pharmaceutical agent, Notch, Notch analog, biologically active Notch fragment, Notch fusion protein or nucleic acid sequence is that amount, or dose, administered to an animal that is required to effect a change (increase or decrease) in Notch protein expression or in Notch function. The dosage administered to an animal, including frequency of administration, will vary depending upon a variety of factors, including pharmacodynamic characteristics of the particular augmenting agent, mode and route of administration; size, age, sex, health, body weight and diet of the recipient; nature and extent of symptoms being treated or nature and extent of the cognitive function(s) being enhanced or modulated, kind of concurrent treatment, frequency of treatment, and the effect desired.

Pharmaceutical agents, Notch, Notch analogs, biologically active Notch fragments, Notch fusion proteins and nucleic acid sequences can be administered in single or divided doses (e.g., a series of doses separated by intervals of days, weeks or months), or in a sustained release form, depending upon factors such as nature and extent of symptoms, kind of concurrent treatment and the effect desired. Other therapeutic regimens or agents can be used in conjunction with the present invention. Adjustment and manipulation of established dosage ranges are well within the ability of those skilled in the art.

Once an effective amount has been administered, a maintenance amount of a pharmaceutical agent, Notch, Notch analog, biologically active Notch fragment or Notch fusion protein, or nucleic acid sequence encoding a Notch, Notch analog, biologically active Notch fragment or Notch fusion protein, can be administered to the animal. A maintenance amount is the amount of pharmaceutical agent, Notch, Notch analog, biologically active Notch fragment or Notch fusion protein (or nucleic acid sequence encoding a Notch, Notch analog, biologically active Notch fragment or Notch fusion protein) necessary to maintain the change (increase or decrease) in Notch protein expression or in Notch function achieved by the effective dose. The maintenance amount can be administered in the form of a single dose, or a series of doses separated by intervals of days or weeks (divided doses). Second or subsequent administrations can be administered at a dosage which is the same, less than or greater than the initial or previous dose administered to the animal. Determination of such amounts are well within the ability of those skilled in the art.

The present invention also provides in vitro (e.g., cell-based) and in vivo screening methods for identifying a pharmaceutical agent which modulates or is capable of modulating Notch function by screening for Notch function. In one embodiment, the present invention provides a method for identifying a pharmaceutical agent for its ability to modulate Notch function comprising introducing a pharmaceutical agent of interest into host cells expressing Notch; and determining Notch function in the host cells. A difference in Notch function determined compared to Notch function of the host cells to which the pharmaceutical agent has not been introduced identifies the pharmaceutical agent as a pharmaceutical agent that modulates Notch function. By “capable of modulating Notch function” is meant to include pharmaceutical agents which can modulate Notch function. In one embodiment, Notch function is screened by determining the level of Notch protein expression (translation). As described herein, Notch is transcriptionally upregulated during memory formation. Accordingly, in this embodiment, Notch protein expression can be determined by determining Notch mRNA or protein production. In a second embodiment, Notch function is screened by determining the functional readout of Notch. In this embodiment, a functional readout of Notch can be determined by detecting (such as using an antibody) the translocation of the Notch cytoplasmic domain (Nintra) into the nucleus or by determining the level of Notch protein production. A functional readout of Notch can also be determined indirectly by detecting and measuring downstream gene products regulated by a Notch gene product. In a third embodiment, Notch function is screened by determining presence of the cleaved cytoplasmic domain of Notch (Nintra) in the host cells' nucleus.

By “Notch function” is meant the biological activity of Notch, which includes regulation of expression of genes in the Notch pathway. Biological activity is understood to mean biological function or action. By “Notch pathway function” is meant a set of gene products which interact with a Notch gene product and/or with each other to produce Notch function.

In a particular embodiment, Notch::indicator fusion protein constructs are employed in cell-based screening methods for identifying a pharmaceutical agent which is capable of modulating Notch function. Thus, in one embodiment, a cell-based screening method for identifying a pharmaceutical agent which is capable of modulating Notch function comprises (a) introducing a pharmaceutical agent to be evaluated for its ability to modulate Notch function into host cells expressing a Notch::indicator fusion protein (e.g., Notch:: heat shock protein (N-hs); Notch::GFP fusion protein); and (b) determining Notch function. The Notch function determined in step (b) is compared to the Notch function of the host cells of step (a) to which the pharmaceutical agent has not been introduced (untreated host cells expressing Notch::indicator fusion protein) (control). A difference in Notch function of the treated cells relative to the Notch function of the untreated cells identifies the pharmaceutical agent as one which modulates (or is capable of modulating) Notch function.

In one embodiment, Notch function is determined by detecting and determining the level of Notch::indicator fusion protein expression (translation) (e.g., by detecting Notch::indicator fusion protein mRNA or protein production). In a second embodiment, Notch function is determined by detecting (such as using an antibody) the translocation of the Notch::indicator fusion protein into the host cells' nucleus or by determining the level of Notch (e.g., cleaved cytoplasmic domain of Notch)::indicator fusion protein production. In a third embodiment, Notch function is determined indirectly by detecting and measuring downstream gene products regulated by a Notch gene product.

In another embodiment, a cell-based screening method for identifying a pharmaceutical agent capable of modulating Notch function comprises (a) introducing a pharmaceutical agent to be evaluated for its ability to modulate Notch function into host cells, said cells expressing Notch::indicator fusion protein; and (b) detecting the translocation of the Notch::Indicator fusion protein into the host cell's nucleus. A difference in translocation of Notch::indicator fusion protein into a host cell's nucleus in the presence of a pharmaceutical agent relative to its translocation in the absence of pharmaceutical agent identifies the pharmaceutical agent as one which modulates (or is capable of modulating) Notch function.

Cells expressing a Notch::indicator fusion protein can be produced by introducing into host cells a DNA construct comprising (1) DNA encoding a Notch::indicator fusion protein (e.g., Notch::heat shock protein; Notch::GFP fusion protein); and (2) a promoter sequence of a ubiquitously expressed gene, wherein the promoter sequence is operably linked to the DNA encoding the Notch::indicator fusion protein. In a particular embodiment, the DNA construct is introduced into host cells, e.g., via a vector, which causes the fusion protein to be expressed in the cells. Expression of the Notch::indicator fusion protein can be transient or stable. As used herein, “a promoter sequence of a ubiquitously expressed gene” refers to a promoter sequence of a gene with widespread expression. Examples of ubiquitously expressed genes are known in the art and include the actin gene and the ELAV gene. In another embodiment, an inducible promoter can be used. An inducible promoter refers to a promoter sequence of a gene that expresses the gene in the presence of a condition (e.g., heat shock, trauma) or a substance (e.g., hormone).

A vector, as the term is used herein, refers to a nucleic acid vector, e.g., a DNA plasmid, virus or other suitable replicon (e.g., viral vector). Viral vectors include retrovirus, adenovirus, parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses such as picomavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D-type viruses, HTLV-BLV group, lentivirus, spumavirus (Coffin, J. M., Retroviridae: The viruses and their replication, In Fundamental Virology, 3rd Edition, B. N. Fields, et al., eds., Philadelphia, Pa.: Lippincott-Raven Publishers) (1996)). Other examples include Sindbis virus, murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in McVey et al., U.S. Pat. No. 5,801,030, the teachings of which are incorporated herein by reference.

DNA encoding a Notch::indicator fusion protein can be manufactured as described using methods known and described in the art (see, e.g., Ausubel et al., Current Protocols In Molecular Biology (New York: John Wiley & Sons) (1998); and Sambrook et al., Molecular Cloning: A Laboratory Manual, 2nd edition (New York: Cold Spring Harbor University Press (1989)). DNA constructs comprising a promoter sequence operably linked to DNA encoding a Notch::indicator fusion protein can be manufactured as described using methods known and described in the art (see, e.g, Ausubel et al., Current Protocols In Molecular Biology (New York: John Wiley & Sons) (1998); and Sambrook et al., Molecular Cloning. A Laboratory Manual, 2nd edition (New York: Cold Spring Harbor University Press (1989)). DNA constricts can be introduced into cells according to methods known in the art (e.g., transformation, direct uptake, calcium phosphate precipitation, electroporation, projectile bombardment, using liposomes). Such methods are described in more detail, for example, in Sambrook et al., Molecular Cloning: A Laboratory Manual, 2nd edition (New York: Cold Spring Harbor University Press) (1989); and Ausubel, et al., Current Protocols in Molecular Biology (New York: John Wiley & Sons) (1998).

Anti-Notch antibodies can also be used to determine whether a particular pharmaceutical agent has an effect on Notch function, such as by detecting translocation of Notch protein, as described above. Anti-Notch antibodies can also be used to determine whether a particular pharmaceutical agent has an effect on the levels of Notch protein expression (translation) by detecting Notch protein production.

The present invention also encompasses methods of screening for or identifying a pharmaceutical agent which is capable of modulating Notch function comprising (a) introducing into host cells a DNA construct, wherein the DNA construct comprises (1) DNA encoding an indicator gene; and (2) a Notch promoter sequence operably linked to the DNA encoding the indicator gene; (b) producing a sample by introducing into host cells comprising the DNA construct a pharmaceutical agent to be assessed for its ability to modulate Notch function under conditions appropriate for expression of the indicator gene; (c) detecting and determining the level of indicator gene product produced in the sample obtained in step (b); and (d) comparing the level of indicator gene product detected in step (c) with the level of indicator gene product detected in control cells into which the pharmaceutical agent has not been introduced. A difference in the level of indicator gene product in the sample obtained in step (b) compared to the level of indicator gene product in control cells identifies the pharmaceutical agent as one which modulates Notch function. By “Notch promoter sequence” is meant a promoter sequence usually upstream (5′) of the coding region of the Notch gene, which controls the expression of the coding region by providing recognition and binding sites for RNA polymerase and other factors which may be required for initiation of transcription.

DNA constructs comprising a promoter sequence operably linked to DNA encoding an indicator gene can be manufactured as described using methods known and described in the art. See, for example, Ausubel et al., Current Protocols In Molecular Biology (New York: John Wiley & Sons) (1998); and Sambrook et al., Molecular Cloning. A Laboratory Manual, 2nd edition (New York: Cold Spring Harbor University Press (1989).

The term “indicator gene”, as used herein, refers to a nucleic acid sequence whose product can be easily assayed, for example, calorimetrically as an enzymatic reaction product, such as the lacZ gene which encodes β-galactosidase. Other examples of widely used indicator genes include those encoding enzymes, such as β-glucoronidase and β-glucosidase; luminescent molecules, such as green fluorescent protein (GFP) and firefly luciferase; and auxotrophic markers such, as His3p and Ura3p. See, e.g., Ausubel et al., Current Protocols In Molecular Biology (New York: John Wiley & Sons, Inc.), Chapter 9 (1998)).

As used herein, a “cell” or “host cell” refers to an animal cell. The cell can be a stem cell or somatic cell. Suitable animal cells can be of, for example, mammalian origin. Examples of mammalian cells include human (such as HeLa cells), bovine, ovine, porcine, murine (such as embryonic stem cells), rabbit and monkey (such as COS1 cells) cells. Preferably, the cell is of neural origin (such as a neuroblastoma, neuron, neural stem cell, etc.). The cell can also be an embryonic cell, bone marrow stem cell or other progenitor cell. Where the cell is a somatic cell, the cell can be, for example, an epithelial cell, fibroblast, smooth muscle cell, blood cell (including a hematopoietic cell, red blood cell, T-cell, B-cell, etc.), tumor cell, cardiac muscle cell, macrophage, dendritic cell, neuronal cell (e.g., a glial cell or astrocyte), or pathogen-infected cell (e.g., those infected by bacteria, viruses, virusoids, parasites, or prions).

The cells can be obtained commercially or from a depository or obtained directly from an animal, such as by biopsy.

Pharmaceutical agents which modulate, or are capable of modulating, Notch function can be further screened to determine the effect of the agents on long term memory formation or to identify those agents capable of modulating long term memory. In one embodiment, the method of screening the pharmaceutical agent for its ability to modulate long term memory formation in an animal comprises administering the pharmaceutical agent identified to an animal; and training the animal under conditions appropriate to produce long term memory in the animal. Long term memory in the animal trained is assessed; and compared to long term memory assessed with long term memory produced in a control animal to whom the pharmaceutical agent has not been administered and who has been trained, wherein the pharmaceutical agent is able to modulate long term memory formation in an animal if long term memory formation in the animal is altered when compared to the control animal.

In another embodiment, the method comprises (a) administering to an animal (particularly a mammal) a pharmaceutical agent which modulates, or is capable of modulating, Notch function; (b) training the animal of (a) and a control animal of the same species to which the pharmaceutical agent has not been administered under conditions sufficient to produce long term memory formation in the animals; (c) assessing long term memory formation in the animals trained in step (b); and (d) comparing long term memory formation in the animals assessed in step (c). A control animal is the basis for comparison in assessing results associated with administration of a pharmaceutical agent to an experimental animal. The experimental and control animals are comparable, e.g., same age, genetic makeup, basal Notch function (i.e., Notch function prior to treatment with the pharmaceutical agent). A difference in long term memory formation assessed in the animal treated with (administered) the pharmaceutical agent relative to the long term memory formation assessed in the control animal identifies the pharmaceutical agent as one which has the ability to modulate long term memory formation in the animal.

The present invention also provides methods for screening a pharmaceutical agent for its ability to modulate long term memory formation in an animal and for assessing its effect on long term memory in an animal. In one embodiment, the animal is an adult mammal. In another embodiment, the method of screening a pharmaceutical agent for its ability to modulate long term memory formation in an animal comprises administering the pharmaceutical agent to the animal; and determining the functional Notch gene activity in the animal obtained relative to the functional Notch gene activity in a control animal to whom said pharmaceutical agent has not been administered. The animal having a functional Notch gene activity which differs from the functional Notch gene activity in the control animal to whom said pharmaceutical agent has not been administered is selected; and trained under conditions appropriate to produce long term memory in said animal. Long term memory in the animal trained is assessed; and compared to long term memory produced in the control animal to whom the pharmaceutical agent has not been administered, wherein the pharmaceutical agent is able to modulate long term memory formation in an animal if long term memory in the animal is altered when compared to the control animal to whom the pharmaceutical agent has not been administered.

In another embodiment, the method comprises (a) administering to a first animal a pharmaceutical agent of interest; (b) determining Notch function in the animal administered the pharmaceutical agent in (a) relative to Notch function in a control animal of the same species as the first animal to which the pharmaceutical agent has not been administered; (c) selecting the pharmaceutical agent if the Notch function determined in (b) differs from the Notch function in the control animal; (d) administering the pharmaceutical agent selected in (c) to a second animal; (e) training the second animal administered the pharmaceutical agent in (d) and a control animal of the same species as the second animal under conditions appropriate to produce long term memory formation in the animals; (f) assessing long term memory formation in the animals trained in step (e); and (g) comparing long term memory formation in the animals assessed in step (f). The first and second animals can be of the same or different species. The first animal and the corresponding control animal are comparable, e.g., same age, genetic makeup, basal Notch function (i.e., Notch function prior to treatment with the pharmaceutical agent). Similarly, the second animal and the corresponding control animal are comparable, e.g., same age, genetic makeup, basal Notch function (i.e., Notch function prior to treatment with (administration of) the pharmaceutical agent). A difference in long term memory formation assessed in the animal treated with the pharmaceutical agent relative to the long term memory formation assessed in the control animal identifies the pharmaceutical agent as one which has the ability to modulate long term memory formation in the animal.

The present invention also encompasses methods for screening a pharmaceutical agent for its ability to modulate Notch function in an animal and for assessing the effect of a pharmaceutical agent on Notch function in an animal. In one embodiment, the animal is an adult mammal. In another embodiment, the method comprises (a) administering a pharmaceutical agent of interest to the animal; and (b) determining Notch function in the animal obtained in (a) relative to Notch function in a control animal of the same species to which the pharmaceutical agent has not been administered. The experimental and control animals are comparable, e.g., same age, genetic makeup, basal Notch function (i.e., Notch function prior to treatment with the pharmaceutical agent). A difference in Notch function determined in the animal treated with the pharmaceutical agent relative to Notch function determined in the control animal identifies the pharmaceutical agent as one having the ability to modulate Notch function in the animal.

As used herein, training can comprise one or multiple training sessions and is training appropriate for long term memory formation or for performance of the specified cognitive task. The pharmaceutical agent can be administered before, during or after one or more of the training sessions. In one embodiment, “training” is cognitive training.

Training of mammals for long term memory formation is conducted using methods generally known in the art (see, e.g., Josselyn et al., Society for Neurosci., 24:926, Abstract 365.10 (1998); Guzowski et al., Proc. Natl. Acad. Sci. USA, 94:2693-2698 (1997); Lamprecht et al., J. Neuroscience, 17(21):8443-8450 (1997): Bourtchuladze et al., Cell, 79:59-68 (1994); and Kogan et al., Curr. Biol., 7:1-11 (1996)). In one embodiment, contextual fear conditioning is used. Contextual fear conditioning is a form of associative learning in which animals learn to recognize a training environment (conditioned stimulus, CS) that has been previously paired with an aversive stimulus such as foot shock (unconditioned stimulus, US). When exposed to the same context at a later time, conditioned animals show a variety of conditional fear responses, including freezing behavior (Fanselow, M. S., Behav. Neurosci., 98:269-277 (1984); Fanselow, M. S., Behav. Neurosci., 98:79-95 (1984); and Phillips, R. G. and LeDoux, J. E., Behav. Neurosci., 106:274-285 (1992)). Contextual conditioning has been used to investigate the neural substrates mediating fear-motivated learning (Phillips, R. G. and LeDoux, J. E., Behav. Neurosci., 106:274-285 (1992); and Kim, J. J. et al., Behav. Neurosci., 107:1093-1098 (1993)).

Pharmaceutical agents (drugs), as used herein, are compounds with pharmacological activity and include inorganic compounds, ionic materials, organic compounds, organic ligands, including cofactors, saccharides, recombinant and synthetic peptides, proteins, peptoids, nucleic acid sequences, including genes, nucleic acid products.

Pharmaceutical agents can be individually screened. Alternatively, more than one pharmaceutical agent can be tested simultaneously for the ability to modulate long term memory formation and/or the ability to modulate Notch function in accordance with the methods herein. Where a mixture of pharmaceutical agents is tested, the pharmaceutical agents selected by the methods described can be separated (as appropriate) and identified by suitable methods (e.g., chromatography, sequencing, PCR).

Large combinatorial libraries of pharmaceutical agents (e.g., organic compounds, recombinant or synthetic peptides, peptoids, nucleic acids) produced by combinatorial chemical synthesis or other methods can be tested (see e.g., Zuckerman, R. N. et al., J. Med. Chem., 37:2678-2685 (1994) and references cited therein; see also, Ohlmeyer, M. H. J. et al., Proc. Natl. Acad. Sci. USA, 90:10922-10926 (1993) and DeWitt, S. H. et al., Proc. Natl. Acad. Sci. USA, 90:6909-6913 (1993), relating to tagged compounds; Rutter, W. J. et al. U.S. Pat. No. 5,010,175; Huebner, V. D. et al., U.S. Pat. No. 5,182,366; and Geysen, H. M., U.S. Pat. No. 4,833,092). The teachings of these references are incorporated herein by reference. Where pharmaceutical agents selected from a combinatorial library carry unique tags, identification of individual pharmaceutical agents by chromatographic methods is possible.

Chemical libraries, microbial broths and phage display libraries can also be tested (screened) for the presence of one or more pharmaceutical agent(s) which are capable of modulating long term memory formation and/or modulating Notch function in accordance with the methods herein.

Pharmaceutical agents identified in accordance with the screening methods herein can be administered to an animal to modulate or enhance long term memory formation in accordance with the methods herein. Pharmaceutical agents identified in accordance with the screening methods herein can also be administered in the treatment of the animal with a defect in long term memory formation that is associated with a defect in Notch, wherein the defect is associated with a defect in Notch, in accordance with the methods herein.

As used herein, a defect in long term memory formation associated with a defect in Notch can be a biochemical or developmental defect. The defect in Notch is either a diminution in the amount of Notch produced, a diminution in Notch function of Notch produced or both a diminution in amount of Notch produced and a diminution in Notch function.

“Modulating”, as the term is used herein, includes induction, enhancement, potentiation, reduction, blocking, inhibition (total or partial) and regulation. By “regulation”, as the term is used herein, is meant the ability to control the rate and extent to which a process occurs.

By “enhancing” or “enhancement” is meant the ability to potentiate, increase, improve or make greater or better, relative to a normal (enhancing a wild type) or a defective state (enhancing a defect so that the defect is improved, more like the wild type or improved compared to wild type), a biochemical or physiological action or effect. For example, enhancing long term memory formation refers to the ability to potentiate or increase long term memory formation in an animal relative to the normal long term memory formation of the animal or relative to the long term memory formation in an animal that has a defect in long term memory formation. As a result, long term memory acquisition is faster or better retained.

In the methods of the present invention, Notch intact protein (full length) protein can be used. Alternatively, a functional or biologically active equivalent of intact Notch protein can be used in the methods of the present invention. A functional or biologically active equivalent of intact Notch protein refers to a molecule which functionally resembles (mimics) intact Notch protein. A functional or biologically active equivalent of intact Notch protein need not have an amino acid sequence analogous to the amino acid sequences of the Notch gene products described herein. For example, a functional equivalent of intact Notch protein can contain a “SILENT” codon or one or more conservative amino acid substitutions, deletions or additions (e.g., substitution of one acidic amino acid for another acidic amino acid; or substitution of one codon encoding the same or different hydrophobic amino acid for another codon encoding a hydrophobic amino acid). See Ausubel et al., Eds., Current Protocols In Molecular Biology (New York: John Wiley & Sons) (1997).

As used herein, the term “animal” includes mammals, as well as other animals, vertebrate and invertebrate (e.g., birds, fish, reptiles, insects (e.g., Drosophila species), mollusks (e.g., Aplysia). Preferably, the animal is a mammal. The terms “mammal” and “mammalian”, as used herein, refer to any vertebrate animal, including monotremes, marsupials and placental, that suckle their young and either give birth to living young (eutharian or placental mammals) or are egg-laying (metatharian or nonplacental mammals). Examples of mammalian species include humans and primates (e.g., monkeys, chimpanzees, humans), rodents (e.g., rats, mice, guinea pigs) and ruminents (e.g., cows, pigs, horses). The animal is preferably an adult animal.

### EXEMPLIFICATION

Materials and Methods

Fly Stocks. N^{ts2} were obtained from the Bloomington Stock Center (Bloomington, Ind.) and were outcrossed with FM7 balancer flies for five generations. The presence of the N^{ts2} mutation was confirmed by embryonic lethality at restrictive temperature (30° C.). Transgenic flies, heat-shock N+(hs-N+) and hs-NΔ^{cdc10rpts} were gifts from M. Young's laboratory at The Rockefeller University (New York) (Schroeter, E. H., et al., Nature, 393:382-386 (1998); Schellenbarger, D. L., et al., Genetics, 81: 143-162 (1975)) and were outcrossed for five generations with our standard wild-type strain, w¹¹¹⁸(isoCJ1) (Yin, J. C., et al., Cell, 79:49-58 (1994)). For all behavioral analyses, w¹¹¹⁸(isoCJ1) serves as the control.

Pavlovian Learning and Memory. The training and testing procedures were the same as described in Tully, T., et al., J. Comp. Physiol., 157:263-277 (1985); Tully, T., et al., Cell, 79:35-47 (1994); Yin, J., et al., Cell, 79:49-58 (1994)). Briefly, a group of ≈100 flies was sequentially exposed to two odors (60 s for each with 45 s of rest in between). During exposure to the first odor, flies were simultaneously subjected to electric shock (twelve 1.5-s pulses with 3.5-s intervals, 60 V); this constitutes a single training cycle. To measure “learning,” flies were transferred immediately after training to the choice point of a T maze and forced to choose between the two odors. For 24-h memory, flies were subjected to multiple spaced training sessions (1, 2, or 10 training sessions with a 15-min rest between each) or to massed training (10 sessions with no rest interval). After training, flies were transferred to food vials and stored at 18° C. for 24 h before testing for their distribution in the T-maze arms. The performance index (PI) was calculated on the basis of the distribution of flies in each arm (Tully, T., et al., J. Comp. Physiol., 157:263-277 (1994)). A PI of 0 represented a 50:50 distribution, whereas a PI of 100 represented 100% avoidance of the shock-paired odor. For determining PIs in N^{ts2} at restrictive temperatures, flies were incubated at 30° C. for 2 days, trained, stored during the retention interval, and tested at 30° C.

Sensorimotor Responses. Odor-avoidance responses were quantified by exposing naive flies to each odor (octanol or methylcyclohexanol) versus air in the T maze. After 120 s, the number of flies in each arm of the T maze was counted, and the PI was calculated for each odor individually as reported (Tully, T., et al., J. Comp. Physiol., 157:263-277 (1985)).

The ability to sense and escape from electric shock was quantified by inserting electrifiable grids into both arms of the T maze; shock pulses were delivered to one of the arms. Flies were transported to the choice point of the T maze, allowing them to choose between the two aims. After 60 s, the center compartment was closed, trapping flies in their respective arms. Individual Pls were calculated as for odor acuity. For determining PIs in N^{ts2}, flies were incubated at 30° C. for 2 days, and the measurements were also carried out at 30° C.

Drug Feeding. The drug-feeding protocol is the same as described in Tully, T., et al., Cell, 79:35-47 (1994). Flies were fed 35 mM cycloheximide in 5% glucose dissolved in 3% ethanol at 25° C. for 12 h before heat-shock treatment. After heat-shock treatment, drug was fed for 3 h before training and for 24 h at 18° C. after training during the retention period.

Heat-Shock Treatment. Flies subjected to heat shock were placed in empty vials in a water bath that was maintained at 37° C. for 30 min. Flies then were transferred back to bottles with food and rested for 3 h at room temperature (20-24° C.) before the training sessions. To minimize leaky expression for the groups not subjected to heat shock, flies were incubated at 18° C. overnight before training, which was performed at room temperature.

RT-PCR of Induced N Expression. Flies were raised at either 18 or 25° C. One group of flies was shifted from 18 to 37° C. for 30 min and returned to 18° C. for 3 h before freezing flies at −70° C. Total RNA was prepared from 200 mg of frozen flies by using the RNeasy minikit (Qiagen, Valencia, Calif.), and mRNA was isolated by using the Dynabeads mRNA Direct microkit (Dynal, Madison, Wis.). Three independent RNA preparations were made for each temperature condition. First-strand cDNA was synthesized directly from the mRNA by using the Superscript first-strand synthesis system (Invitrogen). PCR amplification with a 25:1 mixture of Taq DNA polymerase (Invitrogen) and Pfu DNA polymerase (Stratagene) was carried out in Invitrogen 10×PCR buffer using 1.5 mM MgCl₂ with 30 cycles of 94° C. for 1 min, 55° C. for 1 min, and 72° C. for 1 min followed by extension at 72° C. for 10 min. Three pairs of N-specific primers were designed against regions of the Drosophila N cDNA encoding the intracellular domain (GenBank accession no. NM_{—}057511) (Adams, M. D., et al. Science, 287:2185-2195 (2000)). Primers N1 (5′-gatgccaattgtcaggataac-3′) (SEQ ID NO: 1) and N2 (5′-gaatgttcaccgcttcggtat-3′) (SEQ ID NO: 2) amplify a 285-bp fragment between bases 6722 and 7004; primers N3 (5′-agcgaaatggagtcggtcccg-3′) (SEQ ID NO: 3) and N4 (5′-gaattgcttctgctgtgtggca-3′) (SEQ ID NO: 4) amplify a 268-bp fragment between bases 7932 and 8197; primers N5 (5′-ctcggaggcctggagttcggttc-3′) (SEQ ID NO: 5) and N6 (5′-ggatagctatccaacgtttggac-3′) (SEQ ID NO: 6) amplify a 256-bp fragment between bases 8501 and 8754. The N3-N4 and N5-N6 primer pairs span regions of genomic DNA that contain introns, thus allowing distinction between cDNA products and potential genomic contaminants. Lack of genomic DNA contamination was confirmed by the absence of any PCR products when RT-PCR was carried out in control reactions without reverse transcriptase. Ribosomal protein 49 (rp49)-specific primers (5′-atgaccatccgcccagcatac-3′ (SEQ ID NO: 7) and 5′-gagaacgcaggcgaccgttgg-3′ (SEQ ID NO: 8)) were designed to amplify a 391-bp fragment between bases 1 and 391 of the rp49-coding region (GenBank accession no. Y13939). The control rp49 mRNA should be expressed at equal levels in all cells at all stages (O'Connell, P., et al., Nucleic Acids, Res., 12:5495-5513 (1984)).

Results

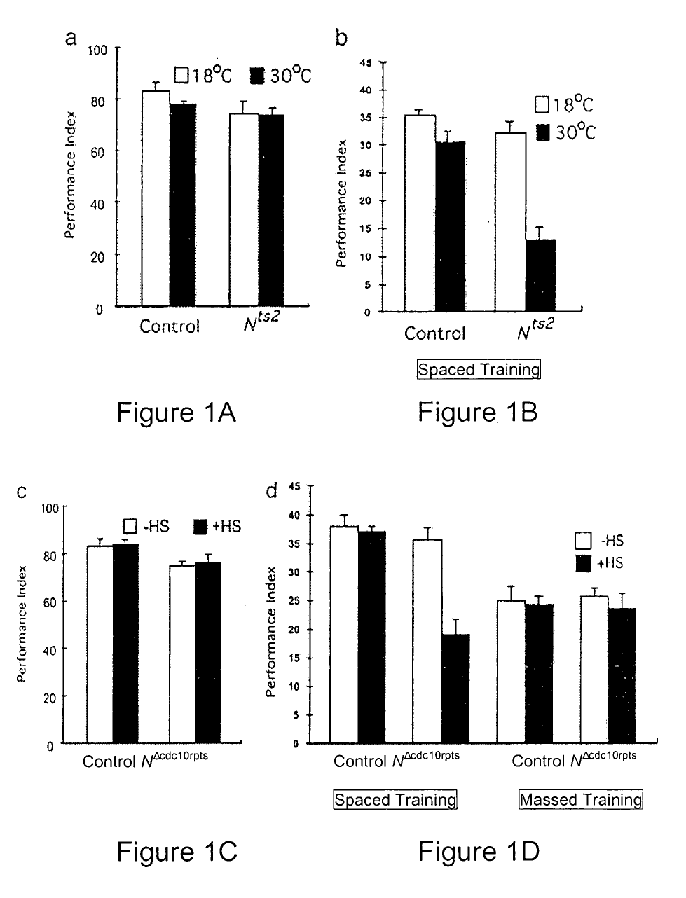

Consistent with the idea that N affects LTM, it was found that alteration of N activity did not affect learning or short-term memory (Ge, X., et al., 101(27):10172-10176 (2004). A temperature-sensitive N allele, N^{ts2}, is viable at permissive temperature but embryonically lethal at restrictive temperature (Lindsley, D. L. & Grell, E. H., eds., Genetic Variations of Drosophila melanogaster (Carnegie Institution, Washington, D.C. (1968)). As adults, these flies were behaviorally normal (shock reactivity and olfactory acuity) (Table) and showed normal learning scores (FIG. 1A) after 2 days of incubation at the restrictive temperature (30° C.). Furthermore, learning was also not affected in transgenic flies that express dominant-negative N, hs-NΔ^{cdc10rpts} (FIG. 1C). Expression of hs-NΔ^{cdc10rpts} was induced acutely via heat-shock treatment (30 min at 37° C., with 3 h of recovery before training; see Materials and Methods) The NΔ^{cdc10rpts} protein lacks the intracellular domain that diffuses into the nucleus after N activation (Schroeter, E. H., Nature, 393:382-386 (1998)). Without the N intracellular domain, the NΔ^{cdc10rpts} protein binds ligands normally but is unable to regulate gene expression (Lieber, T., et al., Genes Dev., 7:1949-1965 (1993)). All mutants tested were outcrossed to control lines to remove potential modifiers.

In contrast, reduction of N activity disrupted 1-day memory. One-day memory after spaced training (10 training sessions with a 15-min rest interval between each) normally is composed of roughly equal amounts of ARM and LTM, whereas 1-day memory after massed training (10 training sessions with no rest interval) is composed only of ARM (Tully, T., et al., Cell, 79:35-47 (1994)). One-day memory was significantly reduced in N^{ts2} mutants at a restrictive temperature (30° C.) when compared with the permissive temperature (18° C.) (FIG. 1B). There was also a slight but statistically insignificant reduction in memory scores in the control group when comparing the higher temperature with the lower temperature (FIG. 1B). Induced expression of dominant-negative N in hs-NΔ^{cdc10rpts} flies also significantly reduced 1-day memory after spaced training (FIG. 1D). Flies were given a 30-min heat shock and subjected to spaced training 3 h afterward. To determine which components (i.e., ARM or LTM) were affected, 1-day memory after massed training in hs-N Δ^{cdc10rpts} transgenic flies were also assayed (FIG. 1D). There was no difference for 1-day memory after massed training, indicating disruption of LTM but not ARM. The heat-shock treatment did not exert any detectable effect on control flies (FIG. 1D). Thus, reduced N activity, resulting either from a conditional mutation or induced expression of a dominant-negative N, specifically blocked the formation of LTM without affecting early memory or ARM.

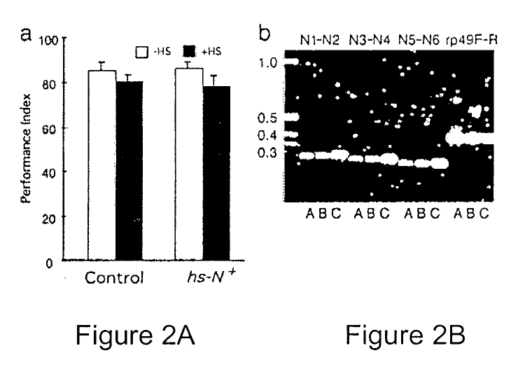

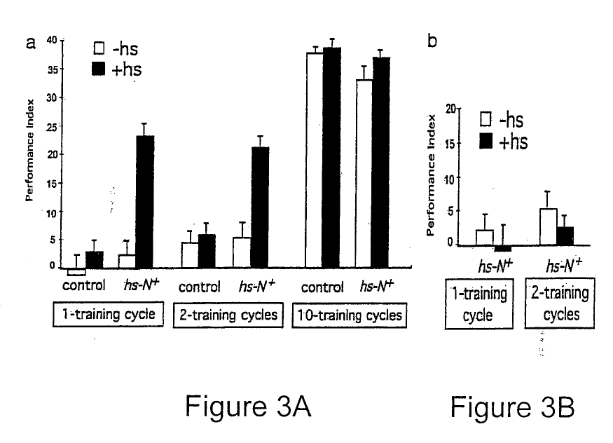

The effects of overexpression of wild-type N⁺ in hs-N⁺ transgenic flies were then evaluated. Flies were given a 30-min heat shock and trained and tested 3 h afterward. Overexpression of N⁺ had no significant effect on learning (FIG. 2A), consistent with our observations that disruption of N does not affect learning. Overexpression of N⁺ after heat-shock treatment was confirmed by RT-PCR of adult head mRNA (FIG. 2B). Acutely induced overexpression of N⁺ also did not affect 1-day memory for flies subjected to the normal spaced training procedure (10 training cycles) (FIG. 3A). However, 1-day memory was enhanced significantly for flies overexpressing N⁺ that had only one or two spaced training sessions (FIG. 3A). To characterize the nature of this enhancement in 1-day memory, it was shown that enhanced memory elicited by one or two spaced training sessions in flies overexpressing N⁺ could be blocked by feeding of cycloheximide, a drug blocking protein synthesis (Tully, T., et al., Cell, 79:35-47 (1994)), to flies (FIG. 3B). It has been shown that feeding cycloheximide to flies can block formation of protein-synthesis-dependent LTM (Tully, T., et al., Cell, 79:35-47 (1994)). There was no significant difference for sensory modalities necessary for performing the learning task (Table). Thus, overexpression of the N⁺ gene facilitates LTM formation.

TABLE
Sensorimotor responses of different genotypes

| Genotype (temp) | OA (MCH) | OA (OCT) | SR |
|---|---|---|---|
| w¹¹¹⁸(isoCJ1) | 70 ± 3 | 69 ± 1 | 85 ± 2 |
| HS-w¹¹¹⁸(isoCJ1) | 73 ± 4 | 67 ± 3 | 84 ± 2 |
| N^{Δcdc10rpts} | 76 ± 4 | 74 ± 4 | 83 ± 1 |
| HS-N^{Δcdc10rpts} | 75 ± 2 | 76 ± 2 | 83 ± 2 |
| hsN⁺ | 74 ± 5 | 73 ± 4 | 83 ± 1 |
| HS-hsN⁺ | 70 ± 5 | 71 ± 5 | 83 ± 2 |
| w¹¹¹⁸(isoCJ1)(30° C.) | 58 ± 6 | 42 ± 9 | 52 ± 7 |
| N^{ts2} (30° C.) | 50 ± 9 | 35 ± 8 | 39 ± 11 |

There is no significant difference for “task-relevant” sensorimotor responses among all comparisons between the control and experimental groups except olfactory acuity for octanol between HS-w¹¹¹⁸ (isoCJ1) and HS-N^{Δcdc10rpts}. All experiments were conducted at room temperature except those indicated as (30° C.). OA, olfactory acuity; MCH, methylcyclohexanol; OCT, octanol; SR, electric-shock reactivity; HS, 37° C. heat shock.
Discussion

In the present study, the data described herein reveals that one function for N in the adult brain is to mediate the formation of LTM. Three different lines of evidence support this conclusion. First, 1-day memory was reduced in a temperature-sensitive N mutant at the restrictive temperature (30° C.) at which N receptor function is supposedly defective. Second, 1-day memory was diminished in transgenic flies that express a dominant-negative N Δ^{cdc10rpts} Third, 1-day memory formation was facilitated by overexpression of N⁺. Although known for its crucial role in development of the nervous system, the use of temperature-sensitive mutations and of transgenic flies carrying inducible genes through acute treatment have allowed the disassociation of the role of N in adult physiology from its role in development. For all experiments that led to the observations noted above, flies were allowed to develop under relatively normal conditions and N function was perturbed acutely at the adult stage before the training.

Moreover, it was also shown that temporary disruption of N function at the adult stage exerted no significant effects on sensorimotor responses as well as on learning. Thus, the observed effects can be attributed specifically to memory, confirming that 1-day memory can be affected by manipulation of N function. The finding described herein is corroborated by an independent observation in which 1-day memory is reduced by disturbing N function through the use of RNA interference and N^{ts1} loss of function in adult flies (Presente, A., et al., Proc. Natl. Acad. Sci, USA, 101:1764-1768 (2004)).

One-day memory consists of both ARM and LTM phases (Tully, T., et al., Cell, 79:35-47 (1994)). The observation described herein indicates a specific effect of manipulation of N function on LTM. One-day memory elicited by massed training (ARM only) (Tully, T., et al., Cell, 79:35-47 (1994)) was not affected by expression of dominant-negative NΔ^{cdc10rpts} whereas 1-day memory after spaced training (ARM and LTM) was reduced significantly. Consistent with this observation, enhanced memory induced by one or two spaced training sessions was blocked by a drug that inhibits protein synthesis. Such drugs are known to block LTM specifically (Tully, T., et al. Cell, 79:35-47 (1994)).

Over the last several decades, a large number of genes in Drosophila have been identified to affect learning and memory through both forward and reverse genetic approaches (for review see Dubnau, J., et al., Ann. Rev. Neurosci., 21:407-444 (1998)). Many signal transduction pathways have been implicated, such as cAMP, Ca²⁺/CaM-dependent kinase, protein kinase C, and mitogen-activated protein kinase pathways, However, none of the components of the N signaling pathway have been implicated by previous studies, which may reflect a crucial role of N signaling in development for which most mutations in this pathway are lethal and therefore unsuitable for behavioral studies.

It is notable that in flies with overexpression of N⁺, only one training session was required to elicit LTM instead of the usual 10 spaced training cycles. This enhancement is very similar to that reported for CREB (Yin, J. C., et al., Cell, 81:107-115 (1995)). Induced expression of hs-dCREb2-activator also reduced the number of spaced training sessions, required to yield the maximal level of LTM in normal flies, from 10 sessions to 1 session (Yin, J., et al., Cell, 81:107-115 (1995)). Involvement of the CREB pathway in LTM formation is a mechanism observed in a wide range of organisms including Drosophila, Aplysia, and vertebrates (Yin, J., et al., Cell, 79:49-58 (1994); Yin, J. C., et al., Cell, 81:107-115 (1995); Bartsch, D., et al., Cell, 83:979-992 (1995); Bourtchuldze, R., et al., Cell, 79:59-68 (1994)). The N signaling pathway is also highly conserved evolutionarily (Artavanis-Tsakonas, S., et al., Science, 284:770-776 (1999)). In fact, a recent report showed that learning and memory are defective in a heterozygous Notch1^{+/-} mouse knockout (Cosat, R., et al., Curr. Biol., 13:1348-1354 (2003)), although a developmental etiology could not be ruled out for such knockout mice. The results described herein reveal an acute role for N signaling during memory formation.

All references cited herein are incorporated herein in their entirety.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of modulating long term memory formation in an animal comprising modulating Notch function in said animal.

2. The method of claim 1 wherein long term memory formation is enhanced.

3. The method of claim 1, wherein modulating Notch function comprises administering to the animal a pharmaceutical agent which modulates Notch function in the animal, in an amount effective to modulate Notch function in the animal.

4. The method of claim 1 wherein modulating long term memory formation in the animal comprises modulating Notch protein expression.

5. The method of claim 4 wherein Notch protein expression is increased thereby enhancing long term memory formation in the animal.

6. The method of claim 4, wherein modulating Notch protein expression comprises administering to the animal a pharmaceutical agent which modulates Notch protein expression in the animal, in an amount effective to modulate Notch protein expression in the animal.

7. A method of enhancing long term memory formation in an animal comprising treating the animal to increase expression of Notch gene relative to expression of the Notch gene in the animal prior to said treatment.

8. A method of treating an animal with a defect in long term memory associated with a defect in Notch comprising increasing Notch function in the animal relative to Notch function in the animal prior to the treatment.

9. The method of claim 8, wherein increasing Notch function comprises administering to the animal a pharmaceutical agent which increases Notch function in the animal, in an amount effective to increase Notch function relative to Notch function in the animal prior to the administering of the pharmaceutical agent.

10. The method of claim 8 wherein Notch protein expression is increased in the animal relative to Notch protein expression in the animal prior to treatment.

11. The method of claim 10 wherein Notch protein expression is increased by administering to the animal a pharmaceutical agent which increases Notch protein expression in the animal, in an amount effective to increase Notch protein expression relative to Notch protein expression in the animal prior to treatment.

12. The method of claim 8 comprising administering to the animal an effective amount of a composition comprising Notch, a Notch analog, biologically active Notch fragment or a Notch fusion protein.

13. The method of claim 8 comprising administering to the animal an effective amount of a composition comprising a nucleic acid sequence which encodes Notch, a Notch analog, biologically active Notch fragment or a Notch fusion protein.

14. A method of treating an animal with a defect in long term memory associated with a defect in Notch gene function comprising treating the animal to increase expression of Notch gene relative to expression of the Notch gene in the animal prior to said treatment.

15. A method of preventing a disease condition in an animal where long term memory is diminished wherein said disease condition is caused by loss of Notch function, by administering a therapeutic amount of Notch protein to an animal in need thereof.

16. The method of claim 15 wherein an inducible form of the Notch gene or an inducible homolog thereof is administered to the animal in need thereof.

17. The method of claim 15 wherein Notch protein expression is increased in the animal relative to Notch protein expression in the animal prior to treatment.

18. A method for identifying a pharmaceutical agent for its ability to modulate Notch function comprising the steps of:
a) introducing a pharmaceutical agent of interest into host cells expressing Notch; and
b) determining Notch function in the host cells of a);
wherein a difference in Notch function determined in step b) compared to Notch function of the host cells to which the pharmaceutical agent has not been introduced identifies the pharmaceutical agent as a pharmaceutical agent that modulates Notch function.

19. The method of claim 18 wherein the host cells of a) express Notch as a Notch::indicator fusion protein.

20. The method of claim 19 wherein Notch function is determined by detecting and determining the level of Notch::indicator fusion protein mRNA produced.

21. The method of claim 19 wherein Notch function is determined by detecting and determining the level of Notch::indicator fusion protein produced.

22. The method of claim 18 wherein Notch function is determined by detecting the presence of the cleaved cytoplasmic domain of the Notch in the host cells' nucleus.

23. The method of claim 18 wherein Notch function is determined by measuring downstream products regulated by the Notch gene product.

24. The method of claim 18 further comprising screening the pharmaceutical agent for its ability to modulate long term memory formation in an animal comprising:
c) administering the pharmaceutical agent identified to an animal;
d) training the animal of c) under conditions appropriate to produce long term memory in the animal;
e) assessing long term memory in the animal trained in step d); and
f) comparing long term memory assessed in step e) with long term memory produced in a control animal to whom the pharmaceutical agent has not been administered and who has been trained, wherein the pharmaceutical agent is able to modulate long term memory formation in an animal if long term memory formation in the animal of step d) is altered when compared to the control animal.

25. A method of screening a pharmaceutical agent for its ability to modulate long term memory formation in an animal comprising the steps of:
a) administering the pharmaceutical agent to the animal;
b) determining the functional Notch gene activity in the animal obtained in step a) relative to the functional Notch gene activity in a control animal to whom said pharmaceutical agent has not been administered;
c) selecting the animal in step b) having a functional Notch gene activity which differs from the functional Notch gene activity in the control animal to whom said pharmaceutical agent has not been administered;
d) training the animal selected in step c) under conditions appropriate to produce long term memory in said animal;
e) assessing long term memory in the animal trained in step d); and
f) comparing long term memory assessed in step e) with long term memory produced in the control animal to whom the pharmaceutical agent has not been administered, wherein the pharmaceutical agent is able to modulate long term memory formation in an animal if long term memory in the animal of step d) is altered when compared to the control animal to whom the pharmaceutical agent has not been administered.

26. A method of screening for or identifying a pharmaceutical agent capable of modulating Notch function comprising the steps of:
a) introducing a pharmaceutical agent of interest into host cells expressing a Notch::indicator fusion protein; and
b) determining Notch function,
wherein a difference in the Notch function determined in b) compared to the Notch function of host cells of a) to which said pharmaceutical agent has not been administered identifies the pharmaceutical agent as one capable of modulating Notch function.
